(19) United States
(12) Patent Application Publication
(10) Pub. No.: US 2006/0089585 A1
(43) Pub. Date: Apr. 27, 2006

(54) Adhesive plaster for treatment of chaps and treatment method thereof

(76) Inventors: Tukasa Takemura, Asaka-shi (JP); Yumi Yoshiura, Yokohama-shi (JP); Kazuki Isobe, Asaka-shi (JP)

Correspondence Address: ADAMS & WILKS;ATTORNEYS AND COUNSELORS AT LAW, 31st FLOOR, 50 BROADWAY, NEW YORK, NY, 10004, US

(21) Appl. No.: 10/974,854

(22) Filed: Oct. 27, 2004

(51) Int. Cl. A61F 15/00; A61F 13/00

(52) U.S. Cl. 602/58; 602/41

(57) ABSTRACT

An adhesive plaster for treatment of chaps formed around the nail of fingertip. On one face of a backing, an adhesive layer is formed, the whole shape is a substantially T-shape, and no pad is provided on this adhesive layer. The backing has a stretchability, and is preferably a non-woven fabric made of elastic fibers, and particularly preferably a non-woven fabric made of ultrafine polyurethane filaments having an average fiber diameter of from about 7 to 15 μm. By using this adhesive plaster, a fingertip having chaps formed is entirely covered closely to treat the chaps.

### TECHNICAL FIELD

The present invention relates to an adhesive plaster for treating chaps. More specifically, it relates to an adhesive plaster which has a configuration particularly suitable for treating chaps formed around the nail of fingertip, and is capable of quickly treating chaps by closing cracks.

### BACKGROUND ART

In the foregoing, first-aid adhesive plasters have been used to treat chaps formed at fingertip by kitchen work, etc. in winter season. However, the first-aid adhesive plaster is used for treatment by putting a pad portion positioned at the center of a rectangular backing onto cracks and protecting and also drying the cracks. Thus, there is a problem that exudation such as blood from the cracks is fixed to the pad after drying, and scabs formed at the cracks are pulled and give pain when the adhesive plaster is peeled. Further, closeness will be insufficient at the portion of a small curved configuration like fingertip, and when water penetrates therein by kitchen work, the pad portion will get wet and the treatment will be delayed.

For the closure of the cracks, as a means for improvement, U.S. Pat. No. 6,410,818 discloses a technique for promoting healing by closing the cracks with an adhesive material. However, the material used for the closure of the cracks described in this publication is of a rectangular shape as well, and this shape is not suitable for enclosing the fingertip in a closed state, and is not suitable for protecting the wound portion around the nail of fingertip. Further, WO 99/16396 discloses an adhesive plaster of a shape suitable for fingertip. The adhesive plaster described in this publication has no pad, and has a shape suitable for enclosing the fingertip, like a boomerang. However, this shape cannot cover the fingertip entirely, and is not suitable for closing the chaps formed at side faces of nail (interface with skin).

### SUMMARY OF THE INVENTION

As described above, the conventional adhesive plaster has problems such that since even a small-size adhesive plaster for fingertip has a pad, it does not fit to a special affected part such as chaps, excess part of a substrate remains at the fingertip and the sticking is not comfortable, and the appearance is bad. In the present invention, in order to solve these problems, no pad is provided and fitting can be improved by use of a substrate having stretchability.

Namely, it is an object of the present invention to provide an adhesive plaster for treatment of chaps and a treatment method thereof, by which even chaps formed around the nail of fingertip can be treated by closing the cracks securely.

The present inventors have found that the above problems can be solved by using an adhesive plaster which is made of a laminate of a stretchable backing and an adhesive and has a substantially T-shape. By forming the adhesive plaster in a substantially T-shape, it fits to the fingertip, and chaps easily formed around the nail of fingertip can be enclosed and protected. Further, when the backing is formed to have stretchability and the adhesive plaster is attached while slightly stretching it, it is possible to entirely close the chaps formed at the front part and right and left parts of the nail, and the adhesive plaster does not easily slip through the fingertip.

As mentioned above, by using the adhesive plaster of the present invention, it is possible to protect the opening of wound, and close it, and treat the chaps in an early stage.

### BRIEF EXPLANATION OF THE DRAWINGS

FIG. 1 is an explanatory view showing chaps formed around the nail of fingertip.

FIG. 2 is a plane view showing an example of an adhesive plaster of the present invention.

FIG. 3 is a plane view showing another example of the present invention.

FIG. 4 is a plane view showing still another example of the present invention.

FIG. 5 is an explanatory view showing a method of using an adhesive plaster for treatment of chaps of the present invention.

FIG. 6 is an explanatory view showing the state where the adhesive plaster is wound around the fingertip by the method shown in FIG. 5.

FIG. 7 is an explanatory view showing another method of use.

FIG. 8 is a bottom view of the adhesive plaster shown in FIG. 2.

FIG. 9 is an enlarged view along the line 9-9 in FIG. 8.

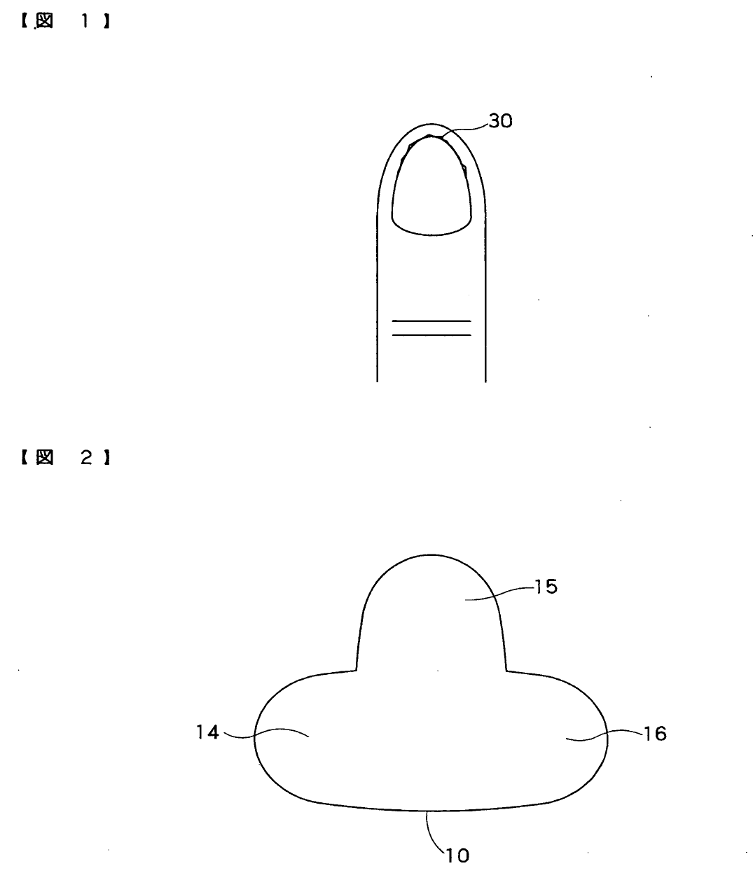

### PREFERRED EMBODIMENTS OF THE INVENTION

The adhesive plaster for treatment of chaps of the present invention comprises a backing 10 having stretchability. As such a stretchable backing, a non-woven fabric made of elastic fibers is preferably used.

As the non-woven fabric made of elastic fibers, non-woven fabrics of polyurethane elastic fibers, polyacryl elastic fibers and other elastic fibers may be used. As the non-woven fabric made of elastic fibers, ones having the following properties are preferably used.

Basic weight 20 to 200 g/m²

Thickness 0.14 to 0.54 mm

10% tensile stress (both longitudinal and transverse directions) 1.0 to 2.2 N/25 mm

100% tensile stress (both longitudinal and transverse directions) 1.6 to 11.8 N/25 mm

100% tensile recovery ratio (both longitudinal and transverse directions) 90%

The basic weight is further preferably about 30 to 150 g/m².

Such non-woven fabric made of elastic fibers is produced by, for example, laminating polyurethane elastic filaments wherein the contact points of the filaments are as if bonded by said filaments by themselves, and may be produced by various methods such as a span bond method, a melt blow method and a flash spinning method. Among them, a non-woven fabric made of elastic fibers produced by a melt blow method is preferred. As the melt blow production method for the non-woven fabric made of polyurethane elastic fibers, for example, a method is proposed wherein a polyisocyanate compound is added to a molten thermoplastic polyurethane elastic material, and it is kneaded, and then spun from orifices and the formed filaments are sprayed along high speed flow and deposited in a sheet-like form on a net conveyor and collected (JP-B-64-8746). In this method, a net conveyor (seamless belt) is moved in the flow direction of the produced non-woven fabric. And, the web collected on the net conveyor is pressed by a roller as the case demands, and wound as a non-woven fabric.

As the fibers of the non-woven fabric, ultrafine filaments having an average fiber diameter of about 7 to 15 μm are preferred. For production of a polyurethane non-woven fabric made of such ultrafine filaments, for example, a method wherein a melt blow method is improved and the diameter of the pore of nozzle is made extremely small, may be mentioned. As commercially available ones for the non-woven fabric made of such ultrafine filaments, Tapyrus (trade name, manufactured by Tonen Tapyrus Co., Ltd.), Espansione (trade name, manufactured by Kanebo Corporation) and the like may be mentioned.

When the fiber diameter of the filaments constituting the non-woven fabric as the backing is made small, an adhesive plaster having a fine texture of the surface and small unevenness in thickness, can be obtained. This non-woven fabric is, of course, excellent in air permeability and stretchability.

If the fiber diameter of the non-woven fabric becomes small, the cooling speed of the fiber becomes high, whereby the fusion by heat at the contact points of respective fibers (filaments) may sometimes be insufficient. In such a case, the internal strength of the non-woven fabric can be enhanced by embossing. As the embossing method, point application method of heat contact bonding method is preferably employed. By such embossing, at least 10 fusion points by heat are formed per 1 cm² so as to make up the insufficiency of internal strength. In addition, by the embossing, slipping properties or the surface of the adhesive plaster (opposite face of an adhesive layer described below) agrees with slipping properties of skin, uncomfortable feeling when an object is grasped by fingers covered with the adhesive plaster will be reduced.

On one face of the backing 10, an adhesive layer 11 is formed (see FIG. 9). As the adhesive, appropriate adhesives of e.g. a natural rubber type, an acrylic type, a silicone type and the like, may be used. Among these adhesives, the acrylic type adhesive is excellent in the adhesion with a polyurethane non-woven fabric and favorable in view of costs, such being preferred. This adhesive layer 11 is obtained by coating the adhesive on one face of the backing 10.

It is further preferred to use an adhesive having a moisture permeability and an air permeability. Further, when the adhesive has no moisture permeability and air permeability, this adhesive may be coated in a frame-like form with a constant width on a peripheral portion of the backing, and partially applied inside the frame with a dot-like pattern, a stripe-like pattern or the like.

The upper face of the adhesive layer 11 is desirably covered by a release liner 12. As the release liner 12, ones obtained by applying a treatment with a release agent such as a silicone resin on paper, a polyester film, a polyolefin film or the like.

A raw fabric of an adhesive plaster obtained by laminating a backing/an adhesive layer/a release liner by a conventional method, is punched out in a desired shape with a punching blade, etc., to prepare the adhesive plaster of the present invention.

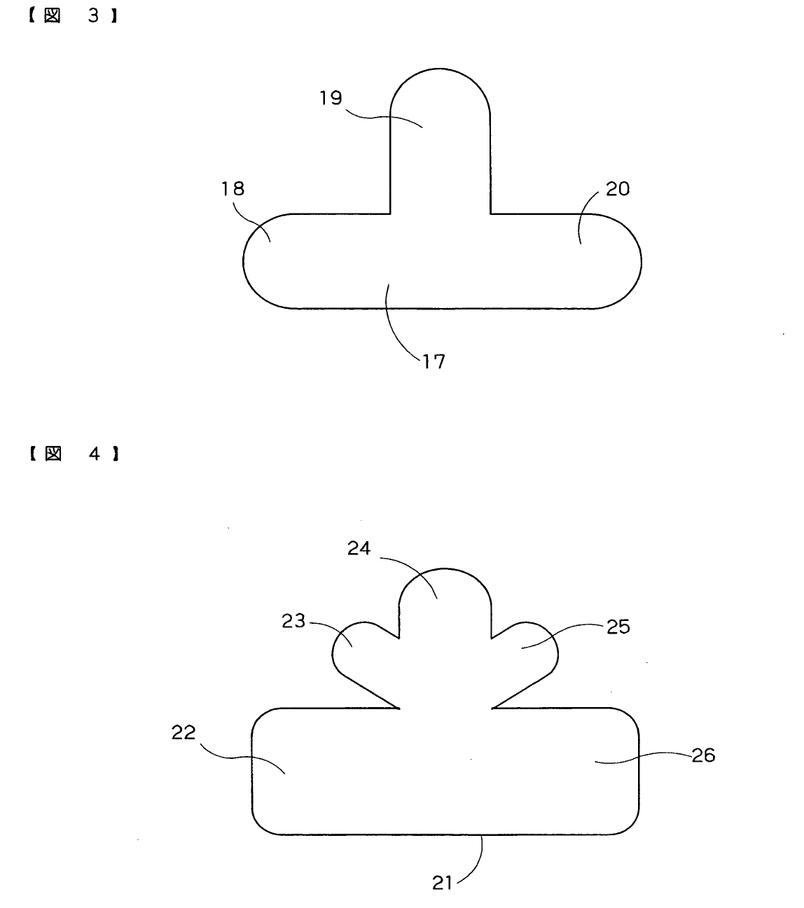

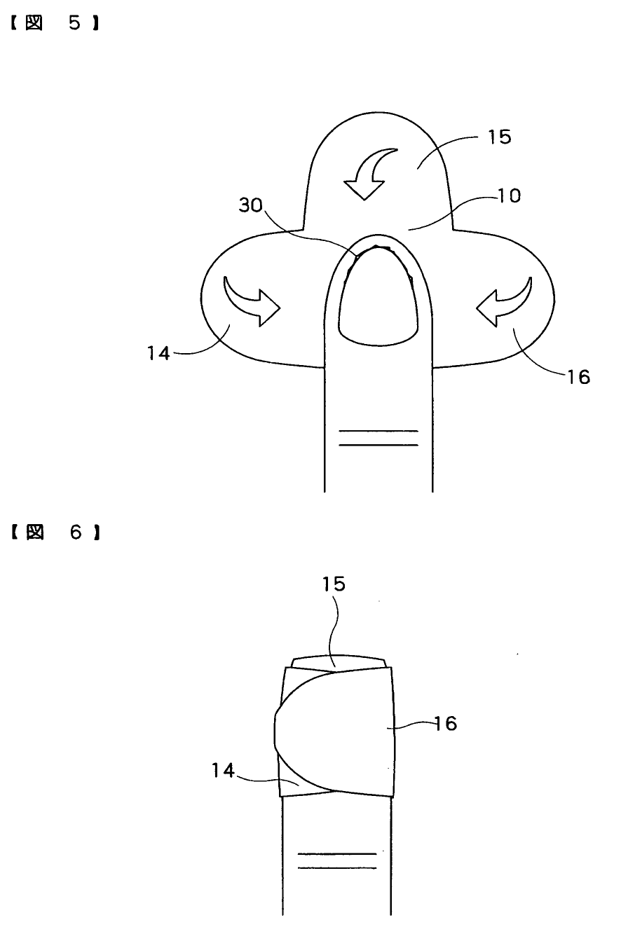

The backing 10 of the adhesive plaster of the present invention has a configuration of a substantially T-shape, by which this can be easily attached to fingertip. Of the backing 10 shown in FIG. 2, protruding portions 14, 15 and 16 towards three directions, respectively, are contoured by rounded outlines. Of the backing 17 shown in FIG. 3, protruding portions 18, 19 and 20 towards three directions, respectively, are substantially liner and the front end portions thereof are contoured in a round shape. By forming the plaster in a substantially T-shape as mentioned above, excessive part will be small at the time or attachment, and the plaster can be fitted to the fingertip more comfortably when attached, and it is excellent from the viewpoint of design. Other configuration of the backing 21 having protruding portions 22, 23, 24, 25 and 26 provided as shown in FIG. 4, may be mentioned. The three protruding portions of the convex shape may be in a shape of rectangular, circular, oval, triangle or the like.

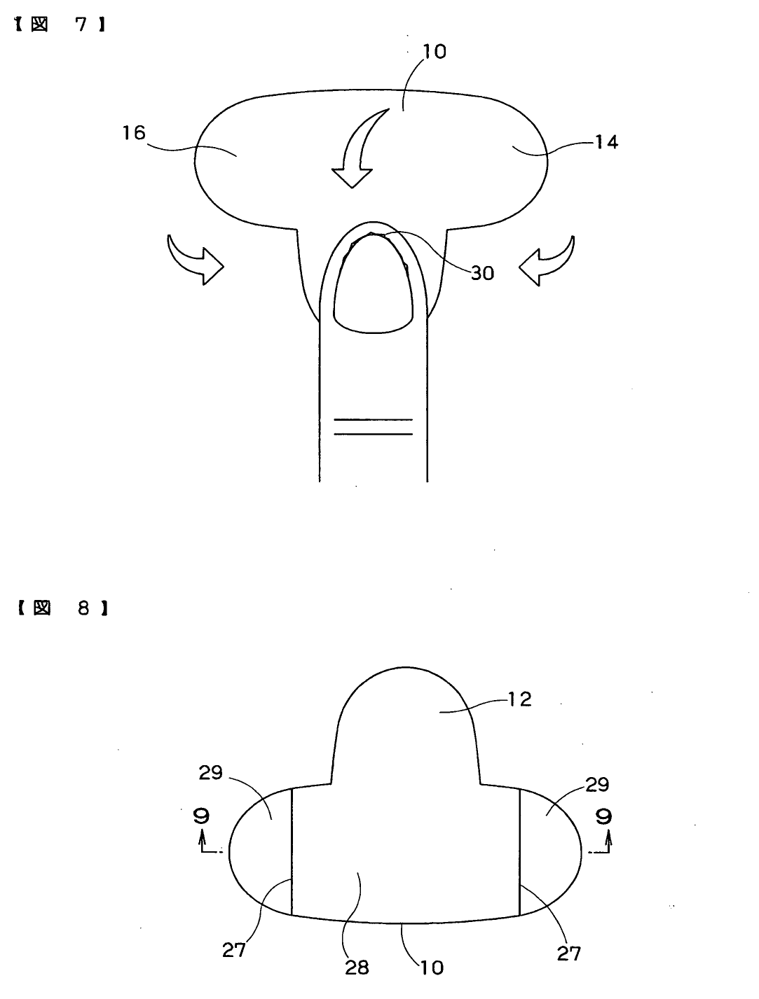

When such an adhesive plaster of the present invention is used, in general, the release liner 12 is peeled, the protruding portion 15 is firstly attached as shown in FIG. 5, and then the protruding portions 14 and 16 on both sides are attached to the fingertip. When the adhesive layer is attached by pressing it onto an affected part, the adhesive layers will not attach to each other. When respective protruding portions 14, 15 and 16 are attached while stretching them a little, since the adhesive plaster has an appropriate stretchability in its entirety, it adapts to the surface of the affected part even if the affected part has concave and convex, no wrinkles are formed, and the plaster can be attached or wound well. It is thereby possible to press the cracks 30 of chaps formed around the nail as shown in FIG. 6 so that the cracks can securely be closed, whereby treatment can be completed in an early time.

Similar effects can be obtained when as shown in FIG. 7, a finger is put on the protruding portion 15, the backing 10 is folded, and then the protruding portions 14 and 16 are attached thereto. As mentioned above, similar effects can be obtained irrespective of the side at which the protruding portions 14 and 16 are overlaid i.e. these are overlaid on the nail side of the finger or overlaid on the thick side of the finger.

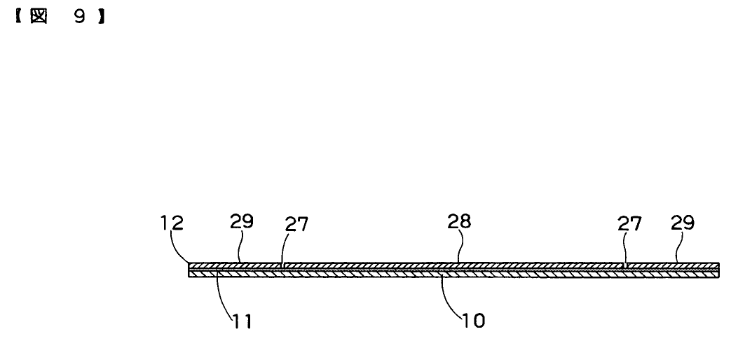

The adhesive plaster can be conveniently used when it has a slit 27 in the release liner 12 as shown in FIGS. 8 and 9, and preferred is to have slits at two portions. When it has slits at two portions, firstly a release liner 28 at the center is removed and the finger is put on the adhesive layer on the central zone. Then, the fingertip is covered and enveloped with the upper convex portion, and release liners 29, 29 at the remaining side zones at both sides are peeled, and these zones are attached around the nail along the finger.

The adhesive plaster of the present invention has excellent fitting properties and gives less uncomfortable feeling, and the tension by winding is neither tight nor loose and gives appropriate feelings. Further, unlike conventional ones, even if the temperature rises or repeating stretch is applied, it adapts to such change sufficiently and does not slip from the finger. Moreover, peeling from the end portion of the attached adhesive plaster can be suppressed. And, air permeability and moisture permeability are excellent, and unpleasant feeling due to heat and humidity and propagation of various bacteria can be suppressed.

1. An adhesive plaster for treatment of chaps, which comprises a stretchable backing and an adhesive layer laminated on one face of the backing, wherein the laminate has a configuration formed in a substantially T-shape so that a fingertip having chaps formed can be entirely covered closely.

2. The adhesive plaster for treatment of chaps according to claim 1, wherein the stretchable backing is a non-woven fabric made of ultrafine polyurethane filaments having an average fiber diameter of from about 7 to 15 μm.

3. The adhesive plaster for treatment of chaps according to claim 1 or 2, wherein the stretchable backing has a 10% tensile stress of 1.0 to 2.2 N/25 mm in both longitudinal and transverse directions.

4. The adhesive plaster for treatment of chaps according to anyone of claims 1 to 3, wherein the surface of the adhesive layer is covered with a release liner and the release liner has a slit so that the release liner can be peeled separately into a central zone and side zones at both sides thereof of the adhesive layer.

5. A method of treating chaps, which comprises winding an adhesive plaster comprising a stretchable backing and an adhesive layer laminated on one face of the backing, wherein the laminate has a configuration formed in a substantially T-shape, around a fingertip having chaps formed to entirely cover the fingertip closely for treatment of the chaps.

6. The method of treating chaps according to claim 5, wherein the stretchable backing is a non-woven fabric made of ultrafine polyurethane filaments having an average fiber diameter of from about 7 to 15 μm.

7. The method of treating chaps according to claim 5 or 6, wherein the stretchable backing has a 10% tensile stress of 1.0 to 2.2 N/25 mm in both longitudinal and transverse directions.
